(19) United States
(12) Plant Patent Application Publication
(10) Pub. No.: US 2003/0106119 P1
(43) Pub. Date: Jun. 5, 2003

(54) Hybrid tea rose plant named 'Why Not Cherise?'

(76) Inventor: James W. Coiner, La Verne, CA (US)

Correspondence Address: CHRISTIE, PARKER & HALE, LLP, 350 WEST COLORADO BOULEVARD, SUITE 500, PASADENA, CA, 91105, US

(21) Appl. No.: 10/016,074

(22) Filed: Dec. 5, 2001

(51) Int. Cl. A01H 5/00

(52) U.S. Cl. PLT/139

(57) ABSTRACT

A new and distinct Hybrid Tea Rose plant variety with dark orange-red flowers.

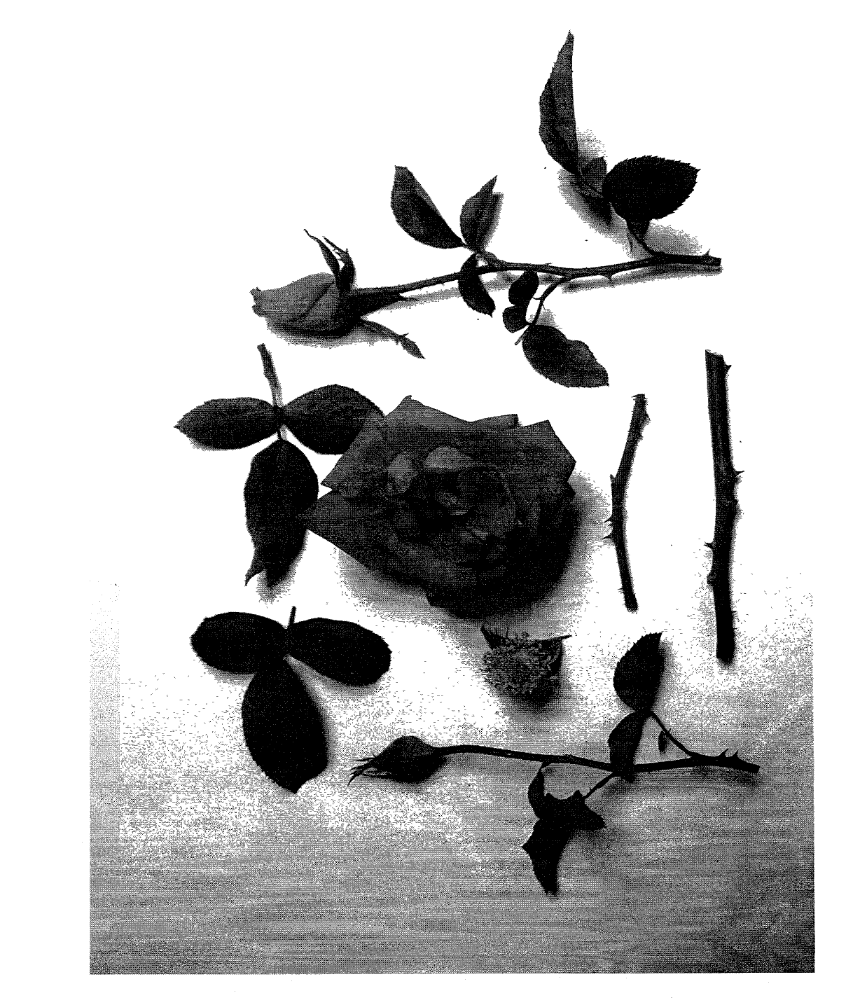

### BACKGROUND OF THE INVENTION

The present invention relates to a new and distinct variety of rose plant of the hybrid tea rose which was created by crossing as seed and pollen parents two (2) unknown rose varieties. The varietal denomination of the new variety is ‘WHY NOT CHERISE?’.

### SUMMARY OF THE INVENTION

Among the novel characteristics possessed by this new variety which distinguishes it from other varieties of which I am aware, is its dark orange-red flowers.

Asexual reproduction by propagation by budding of the new variety as performed in Wasco, California shows that the foregoing and other distinguishing characteristics come true to form and are established and transmitted through succeeding propagations.

### BRIEF DESCRIPTION OF ILLUSTRATION

The accompanying illustration shows typical specimens of the flowers of the new variety depicted in color as nearly true as it is reasonably possible to make the same in a color illustration of this character.

### DESCRIPTION OF THE NEW VARIETY

The following is a detailed description of 2-year budded plants of the new variety, with color terminology in accordance with The Royal Horticultural Society Colour Chart (RHSCC). The terminology used in color description herein refers to plate numbers in the aforementioned color chart. Phenotypic expression may vary with light availability, environmental and cultural conditions.

The following observations are made of specimens grown outdoors in Wasco, Calif., during the month of November.

### PLANT

Form: Upright

Growth: Very vigorous

Size: Height about 5 feet; width about 3 feet p0 Foliage

Size.—Length about 3 inches; width about 1½inches

Quantity.—About 5

Color.—New foliage: Upper side near 146B; under side near 182C Mature Upper side near 147A; under foliage: side near 147C

Shape.—Elliptic

Texture Upper side smooth.—under side matte

Edge.—Serrated

Serration.—Serrate

Petioles.—Top surface color near 146B; Under side near 147C

Stipules.—Medium

Auricle.—Small, slightly serrated

Rachis.—Smooth

Variegation.—None

Veination.—None

### WOOD

New shoots

Bark.—Smooth

Wood.—Near 46A

Old wood

Bark.—Smooth

Wood.—Near 137C

Flowering stem: Length between 4½to 6 inches

Winter Hardiness: Completely winter hardy

Resistance to Disease: Fair

Temperature range: Between 16° and 120° F.

Internode spacing: Usual

Preferred growingl

conditions: Full sun

Growth habit: Upright

Leaves per stem: About 7

### FLOWER/INFLORESCENCE

Blooming Habit: Continuous

Bud About.—1½inches Form: High centered

Color.—Before sepals divide color near 46B; when sepals first divide color near 47B; when petals begin to unfurl color near 47D

Sepals.—Regular; thin on end, thicker on tip

Size.—Length in relation to bud about 1⅜inches

Color.—Topside near 158A; bottom near 147B

Peduncle.—Length: About 4½inches Strength: Erect Color: Near 139D

Flower:

Size.—About 5⅔inches

Borne.—Single

Form.13 Very high centered

Petalage: About 42

Petaloids in center.—Few; between 3 to 4

Persistence.—Good

Fragrance: Medium

Lasting Quality: About 3 weeks on plant; about 7 days after cutting

Color

During first 2 days.—Inner petals at flower center topside near 46C; bottom near 46D; outer petals topside near 47D, bottom near 48C

Base of petals (Point of attachment): Near 2A

General tonality from a distance: Near 4C

Color Change: Fades slightly; after 3 or more days fades to near 49A

Petals

Texture.—Thick

Shape.—Wider than long

Arrangement.—Imbricated

Appearance.—Velvety front/satiny back

### REPRODUCTIVE ORGANS

Arrangement of stamens, Regularly arranged around

Filaments and Anthers: styles

Color: Filaments near 48A; Anthers near 48C

Pollen: No pollen

Styles: Medium length; color near 47C

Hips/Seed Vessels/Ovaries:

Shape.—Round

Size.—Small; about ½inch

Color.—Near 140A

Seeds: No seeds

I claim:

1. A new and distinct Hybrid Tea Rose plant of the variety substantially as shown and described.
